(19) United States
(12) Patent Application Publication
(10) Pub. No.: US 2016/0175229 A1
(43) Pub. Date: Jun. 23, 2016

(54) MICROPARTICLES WITH CYCLODEXTRINS HAVING A DUAL LEVEL OF ENCAPSULATION

(71) Applicants: LCA DERMATECH, Lyon (FR); CENTRE NATIONAL DE LA RECHERCHE SCIENTIFIQUE, Paris (FR); UNIVERSITE CLAUDE BERNARD, Villeurbanne (FR)

(72) Inventors: Charlotte DELALANDE, Lyon (FR); Frédéric BURTIN, Lyon (FR); Yves CHEVALIER, Vernaison (FR); Marie-Alexandrine BOLZINGER, Lyon (FR); Jocelyne PELLETIER, Lyon (FR)

(21) Appl. No.: 14/896,943

(22) PCT Filed: Jun. 16, 2014
(86) PCT No.: PCT/FR2014/051474

§ 371 Date: Dec. 9, 2015

(30) Foreign Application Priority Data
Jun. 14, 2013 (FR) 13/55520

(51) Int. Cl. A61K 8/73; A61K 8/60; A61Q 19/02; A61K 8/67; A61Q 19/00; A61Q 19/08; A61K 8/02; A61K 8/49

(52) U.S. Cl. CPC A61K 8/738; A61K 8/0283; A61K 8/602; A61K 8/4913; A61K 8/678; A61Q 19/00; A61Q 19/08; A61Q 19/02; A61K 2800/56

(57) ABSTRACT

Microparticles include a solid and porous matrix which includes at least one biocompatible polymer. The matrix includes, within its pores, water and at least one inclusion complex formed between a cyclodextrin and at least one first active ingredient. The microparticle is characterized in that the first active ingredient is selected in the group constituted by cosmetic active ingredients and dermatological active ingredients for topical use and in that the microparticle further includes at least one second cosmetic or dermatological active ingredient for topical use which is not in the form of an inclusion complex with the cyclodextrin.

The present invention concerns microparticles of biocompatible polymers which comprise inclusion complex of cyclodextrins with active ingredients, a method for producing these microparticles, as well as their use in the cosmetic and dermatological fields for topical use.

In order to protect and stabilize sensitive active ingredients, it is known to encapsulate them within microparticles of polymers. More specifically, these microparticles may be formed from one porous solid polymer matrix. In the case where the microparticles are intended for cosmetic applications, these polymers are biocompatible polymers.

Preferably, the biocompatible polymers consist of poly(lactic acid) (or polylactide, hereinafter, abbreviated to <<PLA>>), polycaprolactone (hereinafter, abbreviated to <<PCL>>) or poly(lactic-co-glycolic acid) (hereinafter, abbreviated to <<PLGA>>).

Moreover, it is also known to form inclusion complexes between the active ingredients and the cyclodextrin molecules.

Cyclodextrins are cyclic polysaccharides which are linked together so as to form a cavity at their center. This particular form allows the cyclodextrin to receive molecules within its cavity in the form of an inclusion complex. Cyclodextrins are amphiphilic molecules: their exterior is hydrophilic whereas their interior (the cavity) is hydrophobic. Depending on the number of glucoside units that are linked together, there are several sizes of cyclodextrins. The three main sizes are α, β, γ which differ by the radii of their inner cavity (respectively, 5, 6 and 8 Angströms).

Hence, the cyclodextrin can receive a molecule (for example, an active ingredient), thanks to hydrophobic interactions between the inside of the cavity and all or part of said molecule.

It is also possible to functionalize the glucose units of the cyclodextrins, in order to offer new possibilities of interactions with other molecules which require specific interactions so as to form the inclusion complex. For example, it is possible to add hydroxypropyl or methyl functions.

In the context of the present invention, the expressions <<the active ingredient is in the form of an inclusion complex with a cyclodextrin>> and <<the active ingredient is complexed with a cyclodextrin>> are equivalent and mean that the cyclodextrin receives, within its cavity, the active ingredient in the form of an inclusion complex, thanks to appropriate interactions between the cyclodextrin and the active ingredient, which interactions are detailed above. These appropriate interactions may involve all or part of the structure of the cyclodextrin and/or the active ingredient.

Cyclodextrins are biodegradable: their degradation in the organism takes place through slow hydrolysis accompanied with a release of glucose.

The interest of cyclodextrins is multiple:
They allows solubilizing, in water, active ingredients that are not water-soluble;
They stabilize active ingredients;
They protect active ingredients that are sensitive to the external environment, for example to chemical interactions such as hydrolysis and oxidation, or enzymatic or photochemical degradations;
They allow vectorizing active ingredients toward a specific location (in other words, toward a site of action) of an organism and they allow for a release which is controlled over time.

The Patent Application FR 2 944 700 A1 describes a method for forming emulsions which are based, on the one hand, on cyclodextrins and/or hydrophilic polymers carrying cyclodextrins, and on the other hand, hydrophobic compounds, which emulsions present a remarkable stability. The emulsions that are obtained in this manner are used as a cosmetic, food, agricultural or pharmaceutical composition. After administration, these compositions offer the advantage of releasing, over a long period of time, the hydrophobic compound which forms an inclusion complex with the cyclodextrins. Thus, it is possible to perform, by means of these compositions, the administration of the active hydrophobic compound in a controlled manner over time, in particular in the case of an intravenous administration.

The publication of Gupta et al., entitled <<PLGA microparticles encapsulating prostaglandin E1-hydroxypropyl-β-cyclodextrin (PGE1-HPβCD) complex for the treatment of pulmonary arterial hypertension (PAH)>> Pharm. Res. (2011) 28, 1733-1749 describes effectiveness and viability tests of PLGA microparticles which encapsulate an inclusion complex of PEG1 (PEG1 being the abbreviation of prostaglandin E1) and HPβCD (HPβCD being the abbreviation of hydroxypropyl-β-cyclodextrin), in view of the pulmonary release of PEG1 for the treatment of pulmonary arterial hypertension. In this publication, the active ingredient PEG1, which is enclosed by the cyclodextrin, is a pharmaceutical active ingredient. These microparticles are administered through the respiratory tracts in view of their direct absorption in the blood. Thus, this publication discloses a pharmaceutical application of these microparticles of polymers which enclose a cyclodextrin/pharmaceutical active ingredient inclusion complex.

Similarly, the publication of Aguiar et al., entitled <<Encapsultion of insulin-cyclodextrin complex in PLGA microspheres: a new approach for prolonged pulmonary insulin delivery>> Journal of Microencapsulation (2004) 21, 533-564 describes a system for prolonged release of insulin in the blood through pulmonary administration which consists of PLGA microspheres which encapsulate an inclusion complex formed between this hormone and a cyclodextrin.

Thus, the aforementioned two publications describe a use of microspheres which encapsulate an inclusion complex formed from a cyclodextrin and from a pharmaceutical active ingredient in view of a targeted and controlled use in the blood.

It should be borne in mind that the pulmonary mucosae presents a high vascularization, a considerable absorption surface, permeability characteristics of the alveoli and a low enzymatic activity, which conditions are quite suitable for an administration through the respiratory tracts of such PLGA microspheres which encapsulate cyclodextrin/pharmaceutical active ingredient complexes.

On the contrary of the pulmonary mucosae which are constituted from viable cells, the cutaneous barrier is essentially ensured by the stratum corneum which is the outermost layer of the epidermis and which is constituted from keratinized dead cells (corneocytes) which are linked by means of a lipid-based matrix. When active ingredients are administered in the lungs, the ingredients pass mainly through the tight junctions between the epithelial cells, whereas when it comes to the skin, they have to cross a lipid-based <<barrier>>. In other words, in the case of a topical application of an active ingredient, the latter crosses different cutaneous barriers having a biological constitution which is completely different from the biological constitution of the pulmonary mucosae.

As a reminder, the skin comprises different layers which are the stratum corneum, the epidermis and the dermis.

The stratum corneum is the outermost portion of the skin and the epidermis in contact with the external environment and it is formed from dead cells (corneocytes) which are surrounded by a continuous lipid-based medium. A cosmetic formulation is deposited over the stratum corneum.

The epidermis, namely the viable epidermis, which is located immediately below the stratum corneum comprises the stratum granulosum, the stratum spinosum and the stratum germinatum. It consists of a cutaneous compartment which is thin and which predominantly contains keratinocytes. In contrast to the dermis, it is avascular.

The dermis is a connective tissue which is separated from the epidermis by the dermal-epidermal junction and which encloses the fibroblasts, which consist of collagen and elastin fibers that are immersed within an extracellular matrix. It is highly-vascularized. By diffusion, it ensures the epidermis nutrition. It plays a major role in healing, body thermoregulation, as well as in the removal of toxins.

In the cosmetics field, it is known to deliver an active ingredient in a controlled manner over time toward a specific site of action (in other words, a targeted layer of the skin in which the active ingredient should exert its effect) thanks to encapsulation techniques involving polymers such as those that have been detailed above or with inclusion complexes which are formed from cyclodextrins.

In this regard, the Application US 2007/0077292 A1 describes the use of liposomes, as well as cyclodextrins as a vectorization system for administrating collagen and/or the hyaluronic acid in the dermis of the skin. In this document, it is specified that the formation of an inclusion complex of collagen with an alpha-cylodextrin provides a collagen-release system which specifically targets the dermis.

However, in this document US 2007/0077292 A1, there is no mention of any combined release of collagen and hyaluronic acid from this vectorization system which involves cyclodextrins. The release system that is described in the Application US 2007/0077292 A1 is designed to specifically release one single active ingredient at once, and in addition, this release takes place only at the dermis. The cyclodextrins do not form inclusion complexes with polymers such as collagen or hyaluronic acid.

Nonetheless, as has been explained above, it might be quite advantageous, in the cosmetics field, to simultaneously target several layers of the skin, and still further, with different active ingredients depending on the targeted layers of the skin, in order that each active ingredient exerts its effect in the desired layer of the skin.

Thus, none of the documents of the related art that has been detailed above does provides a targeted release system which is controlled over time for more than one active ingredient (in other words, a targeted and controlled release system of at least two active ingredients).

None of these documents of the related art does provide a system for releasing at least two active ingredients which would have to be released differently over time (in other words, a release system which controls the release of at least two active ingredients so that they are not released at the same time). This controlled release of at least two active ingredients would be particularly interesting in the case where it is desired that the active ingredients exert their effect at different moments.

The targeted and time-controlled release, in the different layers of the skin, of dermatological active ingredients for topical use would also be perfectly advantageous in certain dermatological applications.

The inventors of the present invention have developed new microparticles of polymers which encapsulate active ingredients, said microparticles offer the following advantages:
a protection and a stabilization of the cosmetic or dermatological active ingredients for topical use which may be sensitive to hydrolysis and/or enzymatic degradation, to oxidation, to UV irradiation.
the active ingredients, which are advantageously different from each other in order to enhance the active potential of these microparticles, are released in a targeted manner based on their effect in the different aforementioned cutaneous layers.
The release of the active ingredients is controlled over time, according to the following three phases:
1^{st} phase: immediate action at the epidermis.
2^{nd} phase: delayed action at the tissue level.
3^{rd} phase: long-term and in-depth action. This is an action at the cellular level.

A first object of the present invention relates to a microparticle which includes a solid and porous matrix, said matrix comprising at least one biocompatible polymer and including, within its pores, water and at least one inclusion complex formed between a cyclodextrin and at least one first active ingredient, which microparticle is characterized in that the first active ingredient is selected in the group constituted by cosmetic active ingredients and dermatological active ingredients for topical use and in that it further comprises at least one second cosmetic or dermatological active ingredient for topical use which is not in the form of an inclusion complex with the cyclodextrin.

Preferably, the pores that the matrix includes are closed. This allows for a better retention of the inclusion complex within the microparticle, in view of a prolonged release of one or several active ingredients that are enclosed in the pores.

The cosmetic active ingredients that the microparticles according to the invention may include (namely, the first active ingredients and the second active ingredients as have been detailed above) may be advantageously selected among active ingredients which have a beneficial effect on the skin, that is to say those which have an anti-ageing effect, an anti-wrinkle effect, an anti-redness effect, a moisturizing effect, a soothing effect, a brightening effect, a plumping effect or still a purifying effect.

The dermatological active ingredients that the microparticles according to the invention may include (namely, the first active ingredients and the second active ingredients as have been detailed above) may be selected among ketoprofen, griseofulvin, antifungals of the imidazole family such as ketoconazole, antifungals of the allylamine family such as terbinafine, antifungals of the polyene family such as amphotericin B.

In the context of the present invention, it is intended by <<antifungal agent>>, an active ingredient for cutaneous use for treating mycoses, candidoses, molds and cutaneous dermatophytoses.

In the context of the present invention, it is intended by <<biocompatible polymer>>, a polymer which is characterized in that nor the polymer or the products of its degradation do present any toxicity or irritancy or immunogenicity.

Preferably, the biocompatible polymer is further biodegradable. It is intended by <<biodegradable polymer>>, a polymer the biochemical degradation of which by the organism is rapid enough in order not to cause any accumulation in the organism.

Advantageously, the cyclodextrin may be selected in the group constituted by α, β, γ cyclodextrins and their derivatives.

The first active ingredient is a molecule the size and nature of which are appropriate for forming an inclusion complex with the cyclodextrin.

In one embodiment of the invention, at least one portion of the chemical structure of the first active ingredient is, or is made, hydrophobic. This hydrophobic portion of the first active ingredient forms an inclusion complex with a cyclodextrin. This hydrophobic portion of the first active ingredient may consist of a saturated or unsaturated carbon chain, or an aromatic group or the combination of both.

In one embodiment of the invention, at least one portion of the chemical structure of the first active ingredient has been functionalized so as to make this portion hydrophobic in order to make it appropriate for forming an inclusion complex with a cyclodextrin. Thus, this first active ingredient, which ingredient includes a functionalized portion of its chemical structure, may be in the form of an inclusion complex with a cyclodextrin, or in other words, said first chemical ingredient may be complexed with a cyclodextrin thanks to the portion of the functionalized portion of its chemical structure.

In another embodiment of the invention, certain glucose units of the cyclodextrin are functionalized, for example with one or several hydroxy-propyl or methyl functions, so that the cyclodextrin forms an inclusion complex with the first active ingredient, in the case where the chemical structure of this first active ingredient is devoid of at least one hydrophobic portion that is likely to form an inclusion complex with a cyclodextrin. This embodiment of the invention may apply to the hydrophilic active ingredients.

Of course, depending on the first active ingredients that the microparticles according to the invention are desired to include, those skilled in the art would know how to functionalize the first active ingredients or the cyclodextrins. In other terms, those skilled in the art know how to implement the appropriate conditions in order that the first active ingredients of the microparticles according to the invention form an inclusion complex with cyclodextrins. They might be lead to adapt the chemical structure (for example, by functionalizing at least one portion of the chemical structure) of the first active ingredients, or still the chemical structure of the cyclodextrins, without any difficulty, in order to obtain inclusion complexes between the first active ingredients and the cyclodextrins.

The microparticles according to the invention may comprise a plurality of inclusion complexes formed from a plurality of cyclodextrins which are identical to each other or different from each other, and from a plurality of first active ingredients which are identical to each other or different from each other.

In other words, the microparticles according to the invention may comprise:
a plurality of inclusion complexes formed from cyclodextrins which are identical to each other, and from first active ingredients which are different from each other;
a plurality of inclusion complexes formed from cyclodextrins which are different from each other, and from first active ingredients which are identical to each other;
a plurality of inclusion complexes formed from cyclodextrins which are different from each other, and from first active ingredients which are different from each other;
a plurality of inclusion complexes formed from cyclodextrins which are identical to each other, and from first active ingredients which are identical to each other.

Preferably, the first active ingredient is selected in the group constituted by:
hesperidin,
the hesperidin derivatives, preferably the alpha-glucosyl-hesperidin and the methyl-chalcone hesperidin,
the lipoic acid,
the lipoic acid derivatives, preferably the Liposol maleate and the dihydrolipoic acid.

These hesperidin and lipoic acid derivatives may be obtained through chemical or biological transformations of the initial molecules. These first active ingredients, which have just been detailed above, include at least one hydrophobic portion with forms an inclusion complex with a cyclodextrin. Thus, the microparticles according to the invention comprise, within the pores of the biocompatible polymer, at least one inclusion complex formed between a cyclodextrin and any of these first active ingredients which have just been detailed above.

Of course, in the context of the present invention, it is also possible to consider other first active ingredients comprising at least one hydrophobic portion which forms an inclusion complex with a cyclodextrin.

In addition, in the context of the present invention, it is also possible to consider first active ingredients at least one portion of their chemical structure has been made hydrophobic in order to form an inclusion complex with a cyclodextrin.

And as has been explained, in the context of the present invention, it is also possible to consider a first active ingredient which includes at least one portion of its chemical structure which has been functionalized so as to make it hydrophobic in order to make it appropriate for forming an inclusion complex with a cyclodextrin.

Preferably, the biocompatible polymer that forms the matrix of the microparticles is selected in the group constituted by PLA, PLGA and PCL. The matrix may be formed from only one of these polymers in the form of homopolymers or copolymers or still in the form of a mixture of these polymers.

As has been explained above, the microparticles according to the invention further comprise at least one second cosmetic or dermatological active ingredient for topical use which is not in the form of an inclusion complex with the cyclodextrin (or in other words, which has not been complexed with the cyclodextrin) in said microparticles according to the invention.

In the case where the second active ingredient is a hydrophobic active ingredient, this ingredient is solubilized in the solid biocompatible polymer matrix. In this context, it is intended by <<hydrophobic active ingredient>>, an ingredient the solubility of which in non-polar organic solvents is significantly higher than its solubility in water. For example, it may consist of tocopherol acetate, menthol, methyl nicotinate, unsaturated fatty acids, retinol, tocopherol and their derivatives.

In the case where the second active ingredient is a hydrophilic active ingredient, that is to say in the case where it is soluble in non-polar organic solvents, it is contained in the pores of the solid biocompatible polymer matrix which, recall, further enclose water and the inclusion complex formed between the cyclodextrin and the first active ingredient. For example, it may consist of caffeine, the pyrrolidone carboxylic acid, amino acids, peptides, oligosaccharides, polysaccharides and their derivatives.

Thus, in the context of the present invention, said second active ingredient may be a hydrophilic active ingredient contained in the pores of the matrix (namely, the biocompatible polymer matrix) and/or a second hydrophobic active ingredient which is solubilized in the matrix (namely, the biocompatible polymer matrix) of the microparticles according to the invention.

In the case where, in one preferred embodiment of the invention, the pores of the matrix are closed, this also allows for a better retention of this second active ingredient within the microparticle and its prolonged release out of these pores.

The second active ingredient may be identical to or different from the first active ingredient.

In fact, in one embodiment of the invention, a given active ingredient which, when in its initial form, cannot be complexed with a cyclodextrin (for example, as it is devoid of any appropriate hydrophobic portion as has been detailed above), may have been functionalized in order to make a portion of its chemical structure hydrophobic so that it forms an inclusion complex with a cyclodextrin. Thus, thanks to this functionalization, this given active ingredient constitutes a first active ingredient in the sense of the present invention. In this embodiment of the invention, the microparticles according to the invention may comprise:
this first active ingredient, which has been functionalized and which is in the form of an inclusion complex with a cyclodextrin in said microparticles according to the invention,
a second active ingredient, identical to the first active ingredient, but which has not been functionalized, and therefore, is not in the form of an inclusion complex with the cyclodextrin of said microparticles.

In one embodiment of the invention, the second active ingredient is an ingredient which cannot form inclusion complexes with a cyclodextrin, whether because its size is not compatible with the size of the cavity of the cyclodextrin or still because it has no hydrophobic portion as described above. However, this second active ingredient may require a protection by encapsulation within a biocompatible polymer particle. The microparticles according to the invention may perfectly encapsulate this type of second active ingredient.

Another object of the present invention relates to a method for producing microparticles as described above.

This method is based on the principle of encapsulation by solvent evaporation, via an <<aqueous-in-organic-in-aqueous>> type double-emulsion, is characterized in that it comprises the following steps consisting in:

a) preparing a first aqueous solution which comprises at least one first active ingredient selected in the group constituted by the cosmetic active ingredients and the dermatological active ingredients for topical use, and at least one cyclodextrin, the amounts of the first active ingredient and the cyclodextrin being determined so that the first active ingredient and the cyclodextrin form an inclusion complex.

b) preparing a second organic solution which comprises at least one organic solvent and at least one biocompatible polymer, the organic solvent solubilizing the biocompatible polymer.

c) introducing the first aqueous solution in the second organic solution and stirring this set, which set results from the combination of these two solutions, so as to obtain an aqueous-in-organic type emulsion.

d) introducing this emulsion which has been obtained upon completion of step c) in a third aqueous solution and stirring this set, which set results from the combination of the emulsion and the third solution, so as to obtain an aqueous-in-organic-in-aqueous type double emulsion.

e) extracting the organic solvent by means of a water-soluble organic co-solvent.

f) evaporating the organic solvents and co-solvents, so as to obtain an aqueous suspension of microparticles as described above.

g) optionally, recovering the above-described microparticles.

In the context of the present invention, depending on the pH conditions that are imposed for maintaining the stability of the first active ingredient, it is eventually possible to adjust the pH of the aqueous solutions with a buffer solution.

The selection of the amounts of the first active ingredient and those of the cyclodextrin that are appropriate for obtaining an inclusion complex is perfectly within the reach of those skilled in the art. Depending on the first active ingredient, those skilled in the art would know how to adapt the amount of cyclodextrin that is required for the formation of the inclusion complex. In particular, they might select among the different cyclodextrins that are available the cyclodextrin that is the most appropriate for realizing these inclusion complexes.

The formation of an inclusion complex requires a cyclodextrin cavity the size of which corresponds to the size of the molecule to be included within. Depending on the type of molecule to include, the selection of the type of cyclodextrin is performed using the stability constants of the inclusion complexes, which tables of values are detailed in the related literature.

For example, the first active ingredient is selected in the group constituted by:
hesperidin,
the hesperidin derivatives, preferably the alpha-glucosyl-hesperidin and the methyl-chalcone hesperidin,
the lipoic acid,
the lipoic acid derivatives, preferably the Liposol maleate and the dihydrolipoic acid.

These hesperidin and lipoic acid derivatives may be obtained through chemical or biological transformations of the initial molecules.

At step a), the first aqueous solution may further comprise at least one second active ingredient, in the case where the second ingredient is a hydrophilic ingredient.

The first aqueous solution may comprise a plurality of inclusion complexes formed from a plurality of cyclodextrins which are identical or different from each other and a plurality of first active ingredients which are identical or different from each other.

At step b), in a preferred manner, the second organic solution comprises an organic solvent such as dichloromethane or ethyl acetate.

In one embodiment of the invention, the second organic solution further comprises at least one second active ingredient, in the case where the second active ingredient is a hydrophobic ingredient.

The biocompatible polymer is immiscible with water. It may be selected in the group constituted by the homopolymers and copolymers of PLA, PLGA, PCL, poly(orthoesters), polyethers, polysaccharides (chitosan and modified celluloses such as ethyl cellulose), polyacrylates and polymethacrylates, as well as the derivatives of these polymers, whether taken separately or in a mixture. A list of examples of such biocompatible polymers, which are appropriate in the context of the invention, is detailed in the publication of Nair and Laurencin, entitled <<Biodegradable polymers as biomaterials>> Prog. Polym. Sci. 2007, 32, 762-798.

Advantageously, the concentration of the biocompatible polymer in the second organic solution is comprised between 1 and 80 weight %, preferably between 5 and 40 weight %.

Preferably, the biocompatible polymer is PLA.

At step c), the mass ratio of the first aqueous solution to the mass of the second organic solution is preferably lower than 50/50, preferably in the range of 30/70.

At step c), in a preferred manner, stirring is performed at a magnitude which is high enough for resulting in the formation of very fine emulsion droplets. For example, such a vigorous stirring is performed by means of a rotor-stator turbine at high rotational speeds of the rotor, the speed may be comprised, for example, between 8000 and 24000 rpm, and for a time period comprised between 15 and 60 seconds. In another embodiment, the first emulsion is obtained by ultrasonic dispersion.

Advantageously, upon completion of step c), the emulsion comprises internal phase droplets the size of which is smaller than 10 μm, preferably comprised between 0.5 and 6 μm.

At step d), the third aqueous solution preferably consists of an aqueous solution which comprises at least one emulsifier. The emulsifier allows stabilizing the microparticles which are suspended in water, so that a biocompatible polymer matrix is formed.

Preferably, the emulsifier is a poly(vinyl alcohol) (hereinafter, abbreviated to <<PVAL>>), which alcohol is advantageously hydrolyzed to 88%. The molar masses of this polymer generally range between 10 000 and 88 000 g/mol.

Polyvinylpyrrolidone is another emulsifier which may be considered in the context of the present invention.

The concentration of the emulsifier of this third aqueous solution may be comprised between 0.5 g/L and 10 g/L.

At step d), the mass ratio of the emulsion that is obtained upon completion of step c) to the mass of the third aqueous solution is lower than 50/50, preferably in the range of 20/80.

At step d), in a preferred manner, stirring is performed at a lower magnitude or for a time period which is shorter than step c). For example, stirring is performed by means of a rotor-stator type turbine, at a speed which may be comprised between 8000 and 24000 rpm, and for a time period comprised between 15 and 60 seconds.

Upon completion of step d), the double emulsion comprises droplets the size of which is at least five times larger than the size of the droplets of the internal aqueous phase of the emulsion which, recall, contains the first active ingredients and, eventually, the second active ingredients in the case where these are hydrophilic. It should be at least five times larger, since this is the smallest size that is required for ensuring that the biocompatible polymer drops properly surround the droplets of the internal aqueous phase of the emulsion, so that pores could form in the polymer matrix, which drops are advantageously closed.

The porosity of the microparticles according to the invention is controlled thanks to the involvement of a double emulsion. As the solvent is being removed, the biocompatible polymer is solidified by precipitation, thereby forming a polymer matrix around the internal aqueous phase which lies therein in a dispersed form. This internal aqueous phase includes the first active ingredients, and eventually the second active ingredients in the case where the second active ingredients are hydrophilic. This aqueous phase is dispersed in the polymer matrix, thereby forming aqueous pores in the latter.

Preferably, step e) is carried out by transferring the double emulsion into a fourth aqueous solution which contains a co-solvent, preferably at a mass concentration ranging from 2 to 10 weight % of the mass of said fourth aqueous solution.

The co-solvent may consist of any other water-miscible solvent, in which solvent the organic solvent may be solubilized in view of its extraction. It may consist of isopropanol, ethanol, acetone or acetonitrile.

The mass concentration of the co-solvent of this fourth aqueous solution is lower than 20% of the mass of this fourth aqueous solution.

Upon completion of step f) of the method, where an aqueous suspension of microparticles according to the invention is obtained, it is possible to add, to this aqueous suspension, at least one third cosmetic or dermatological active ingredient for topical use. The third active ingredient is properly selected so as to exert an immediate effect and based on its site of action in the skin. The third active ingredient may consist of a vegetal extract.

An object of the present invention relates to an aqueous suspension comprising microparticles as described above.

Advantageously, said aqueous suspension further comprises at least one third active ingredient as described above.

In the aqueous suspension according to the invention, said third cosmetic or dermatological active ingredient for topical use forms no inclusion complex with the cyclodextrins of the microparticles according to the invention, and in addition, it is not present in the biocompatible polymer matrix nor is it present in the pores of said microparticles according to the invention. In other words, said third active ingredient, which ingredient may be comprised in the aqueous suspension according to the invention, is not encapsulated in the microparticles according to the invention. In other terms, the third active ingredient is not located inside the microparticles according to the invention.

Another object of the present invention relates to a cosmetic composition which comprises at least one aqueous suspension of microparticles as described above.

In preferred embodiments of the invention, said cosmetic composition consists of a water-in-oil type or an oil-in-water type emulsion, multiple emulsions such as water-in-oil-in-water type or oil-in-water-in-oil type emulsions, hydrodispersions, lipodispersions, gels, or any other galenic form which is known by those skilled in the art. Depending on the active ingredients that the cosmetic composition comprises, it may consist of a facial serum, a face-and-neck vanishing cream, a cream for the eye contour area, a protective emulsion or a facial lotion, a body care product.

The microparticles recovery step g) may be carried out by centrifugation and filtration. More specifically, the microparticles, which are suspended in the aqueous solution, are centrifuged, preferably at a speed in the range of 10000 rpm for at least 10 minutes. Thus, the supernatant is removed.

Another object of the invention relates to a cosmetic composition which comprises microparticles as described above. In one embodiment of the invention, this cosmetic composition further comprises at least one third cosmetic active ingredient, preferably a third cosmetic active ingredient as described above. This third active ingredient provides additional properties to the cosmetic composition according to the invention.

In preferred embodiments of the invention, said cosmetic composition consists of a water-in-oil type or an oil-in-water type emulsion, multiple emulsions such as water-in-oil-in-water type or oil-in-water-in-oil type emulsions, hydrodispersions, lipodispersions, gels, or any other galenic form which is known by those skilled in the art. Depending on the active ingredients that the cosmetic composition comprises, it may consist of a facial serum, a face-and-neck vanishing cream, a cream for the eye contour area, a protective emulsion or a facial lotion, a body care product.

Another object of the present invention relates to a dermatological composition for topical use which comprises at least one aqueous suspension of microparticles as described above.

Another object of the present invention relates to a dermatological composition for topical use which comprises microparticles as described above. In one embodiment of the invention, this dermatological composition for topical use further comprises at least one third dermatological active ingredient for topical use, preferably a third dermatological active ingredient for topical use as described above. This third active ingredient provides additional properties to the dermatological composition for topical use according to the invention.

Three embodiments of microparticles according to the invention are described below with the involved amounts, with:
A: the second organic solution;
B: the first aqueous solution;
C: the third aqueous solution;
D: the fourth aqueous solution.

### FIRST EXAMPLE

The microparticles that are obtained upon completion of the method according to the invention and which comprise one single active ingredient, namely a first active ingredient which consists of hesperidin.

### SECOND EXAMPLE

The microparticles that are obtained upon completion of the method according to the invention and which comprise two active ingredients, namely a first active ingredient which consists of hesperidin and a second ingredient which consists of pyrrolidone carboxylic acid (namely a second hydrophilic active ingredient).

### THIRD EXAMPLE

The microparticles that are obtained upon completion of the method according to the invention and which comprise three active ingredients, namely a first active ingredient which consists of hesperidin, two second ingredients which consist of pyrrolidone carboxylic acid and tocopherol acetate (namely a second hydrophobic ingredient).

### A. Steps a) to c) of the Method According to the Invention, Upon which Steps an Emulsion is Obtained

### FIRST EXAMPLE

| | | Fraction of | |
|---|---|---|---|
| | | the | |
| | | ingredient in | Weight |
| | | the | percentage in |
| | | considered | the emulsion |
| PHASE | Ingredient | phase (mL) | (%) |
| A | poly(lactic acid) | 2 | 33 |
| | dichloromethane | 3 | 50 |
| B | activated water | 1 | 17 |
| | Example 1: | | |
| | namely | | |
| | hesperidin | 0.25 | 4 |
| | beta-cyclodextrin | 0.02 | 0.3 |
| | water | 0.73 | 12.7 |
| | Total: | 6.00 | 100.00 |

Table 1 detailing, for the first example, the amounts of the constituents which are required for carrying out step a) of the method according to the invention.

### SECOND EXAMPLE

| | | Fraction of | |
|---|---|---|---|
| | | the | |
| | | ingredient | Weight |
| | | in the | percentage |
| | | considered | in the |
| PHASE | Ingredient | phase (mL) | emulsion (%) |
| A | poly(lactic acid) | 2 | 33 |
| | dichloromethane | 3 | 50 |
| B | activated water | 1 | 17 |
| | Example 2: | | |
| | namely | | |
| | hesperidin | 0.25 | 4 |
| | pyrrolidonecarboxylicacid | 0.25 | 4 |
| | beta-cyclodextrins | 0.02 | 0.3 |
| | water | 0.48 | 8.7 |
| | Total: | 6.00 | 100.00 |

Table 2 detailing, for the second example, the amounts of the constituents which are required for carrying out step a) of the method according to the invention.

### THIRD EXAMPLE

| | | Fraction of | |
|---|---|---|---|
| | | the | |
| | | ingredient | Weight |
| | | in the | percentage |
| | | considered | in the |
| PHASE | Ingredient | phase (mL) | emulsion (%) |
| A | poly(lactic acid) | 2 | 33 |
| | dichloromethane | 3 | 50 |
| B | activated water | 1 | 17 |
| | Example 3: | | |
| | namely | | |
| | hesperidin | 0.25 | 4 |
| | pyrrolidonecarboxylicacid | 0.25 | 4 |
| | beta-cyclodextrins | 0.02 | 0.3 |
| | tocopherolacetate | 0.01 | 0.2 |
| | water | 0.47 | 8.5 |
| | Total: | 6.00 | 100.00 |

Table 3 detailing, for the third example, the amounts of the constituents which are required for carrying out step a) of the method according to the invention.

### B. Step d) of the Method According to the Invention, Upon which Step a Double Emulsion is Obtained

For each of the three examples: The activated water corresponds, respectively, to example 1, 2 or 3.

| | | Fraction of | |
|---|---|---|---|
| | | the | |
| | | ingredient | |
| | | in the | |
| | | considered | Percentage in the final |
| PHASE | Ingredient | phase (mL) | double emulsion (%) |
| C | poly(vinylalcohol) | 1 | 1 |
| | water | 49 | 88 |
| Emulsion | Emulsion | 6.00 | 11 |
| | namely | | |
| | poly(lactic acid) | 2 | 3 |
| | dichloromethane | 3 | 6 |
| | activated water | 1 | 2 |
| | according to | | |
| | Example 1, 2 or 3 | | |
| | TOTAL | 56 | 100.00 |

Table 4 detailing, for examples 1 to 3, the amounts of the constituents which are required for carrying out step d) of the method according to the invention.

### C. Step e) of the Method According to the Invention (Namely the Extraction of Dichloromethane)

For each of the three examples: The activated water corresponds, respectively, to example 1, 2 or 3.

| | | Fraction of the | |
|---|---|---|---|
| | | ingredient in | |
| | | the considered | Percentage in the |
| PHASE | Ingredient | phase (mL) | final emulsion (%) |
| D | water | 200.00 | 75 |
| | isopropanol | 10.00 | 4 |
| Double | Double Emulsion | 56.00 | 21 |
| Emulsion | namely | | |
| | poly(vinyl alcohol) | 0.50 | 0.2 |
| | water | 49.50 | 18.6 |
| | Poly(lactic acid) | 2 | 0.8 |
| | dichloromethane | 3 | 1.1 |
| | activated water | 1.00 | 0.4 |
| | according to | | |
| | Example 1, 2 or 3 | | |
| | TOTAL | 266.00 | 100.00 |

Table 5 detailing, for examples 1 to 3, the amounts of the constituents which are required for carrying out step e) of the method according to the invention.

D. Step f) of the above-described method has been carried out and, for examples 1 to 3, three aqueous suspensions of microparticles according to the invention have been obtained.

These aqueous suspensions of microparticles according to the invention may be formulated according to the three examples of formulations that are described below:
a facial vanishing cream;
a facial serum;
a facial tonic.

The percentages are expressed as weight %.

Facial Vanishing Cream

TABLE 6
vanishingcream formulation

| | |
|---|---|
| Aqueous suspension of microparticles | 10.00% |
| according to the invention. | |
| Glycerylstearate + PEG-100 stearate | 6.00% |
| Cetylalcohol | 1.00% |
| Stearylalcohol | 1.00% |
| Beeswax | 1.50% |
| Squalane | 3.00% |
| Hydrogenatedpolyisobutene | 3.00% |
| Cetostearyloctanoate | 1.50% |
| Glyceryltricaprylate/tricaprate | 3.00% |
| Dimethicone | 1.00% |
| Ethylhexylmethoxycinnamate | 2.00% |
| Water | qs 100.00% |
| Xanthangum | 0.20% |
| Carbomer | 0.15% |
| Glycerin | 2.00% |
| Neutralizer | qs |
| Preservatives | qs |
| Perfume, Colorants, . . . | qs |

Facial Serum

TABLE 7
facial serum formulation

| | |
|---|---|
| Aqueous suspension of microparticles | 20.00% |
| according to the invention. | |
| Caprylic/capric/succinictriglycerides | 3.00% |
| Ethylhexylmethoxycinnamate | 1.00% |
| Water | qs 100.00% |
| Acrylates/C10-30 alkyl acrylate | 0.50% |
| crosspolymer | |
| Dimethiconecopolyol | 0.50% |
| Glycerin | 5.00% |
| Neutralizer | qs |
| Preservatives | qs |
| Perfume, Colorants, . . . | qs |

Facial Tonic

TABLE 8
facial tonic formulation

| | |
|---|---|
| Aqueous suspension of microparticles | 1.00% |
| according to the invention. | |
| Water | qs 100.00% |
| Denaturedalcohol (Ethanol) | 3.00% |
| Glycerin | 2.00% |
| PEG-7 GlycerylCocoate | 5.00% |
| Citricacid/Sodium citrate | qs pH 6.50 |
| Perfume | 0.20% |
| Polysorbate 20 | 2.00% |
| Preservatives | qs |
| Perfume, Colorants, . . . | qs |

### DESCRIPTION OF THE FIGURES

FIG. 1a schematically represents an inclusion complex of a cyclodextrin with a first active ingredient.

FIG. 1b schematically represents a portion of a microparticle according to the invention which comprises inclusion complexes which have been represented in FIG. 1a.

FIG. 1c schematically represents an aqueous suspension according to the invention comprising microparticles which have been represented in FIG. 1b.

FIG. 2 is a diagram detailing the amount of a first active ingredient which has cumulated in the dermis during 24 hours, which ingredient has been tested in different forms.

FIG. 3 is a graph of the variation of the amount of a first active ingredient in the receiver liquid as a function of time from 0 to 48 hours, which ingredient has been tested encapsulated in microparticles according to the invention and in a free form.

FIG. 4 is a photograph of a microparticle according to the invention, taken by scanning electron microscopy.

FIG. 5 is a graph of the rate of passage of caffeine into the receiver liquid compared to the deposited amount (expressed in %) as a function of time, for samples 1) to 5).

FIG. 6 is a graph of the rate of passage of the alpha-glucosyl-hesperidin into the receiver liquid compared to the deposited amount (expressed in %) as a function of time, for samples 1) to 5).

FIG. 7 is a graph of the rate of passage of tocopherol acetate into the receiver liquid compared to the deposited amount (expressed in %) as a function of time, for samples 1) to 5).

FIG. 8 is a graph of the rate of passage of tocopherol acetate, caffeine and the alpha-glucosyl-hesperidin compared to the deposited amounts (expressed in %) as a function of time, for samples 1) to 2).

FIG. 9 is a diagram of the relative permeation rates, calculated for the hydrophilic active ingredients which consist of caffeine and the alpha-glucosyl-hesperidin and related the tocopherol acetate, for samples 1) to 5).

FIG. 10 represents a diagram of the rate of passage of caffeine compared to the applied dose and its distribution in the different layers of the skin and in the receiver liquid, after 24 hours, for samples 1) to 5).

FIG. 11 represents a diagram of the rate of passage of the alpha-glucosyl-hesperidin compared to the applied dose and its distribution in the different layers of the skin and in the receiver liquid, after 24 hours, for samples 1) to 5).

FIG. 12 represents a diagram of the rate of passage of tocopherol acetate compared to the applied dose and its distribution in the different layers of the skin and in the receiver liquid, after 24 hours, for samples 1) to 5).

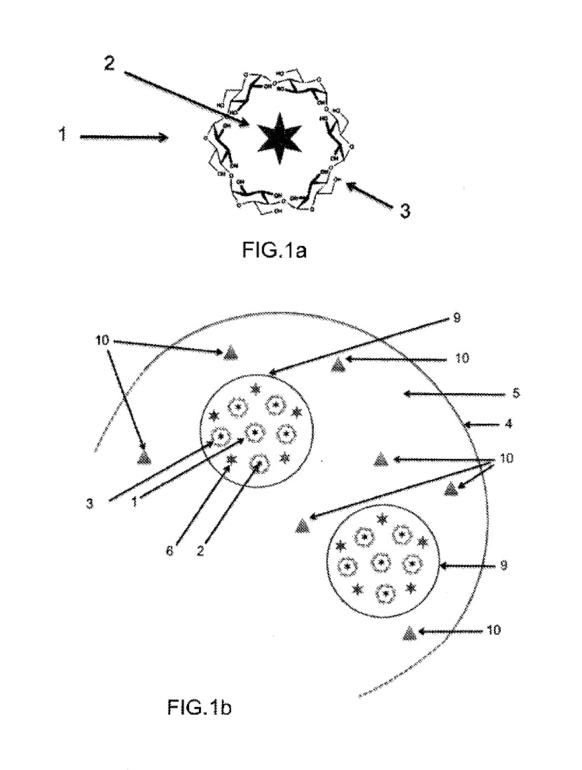

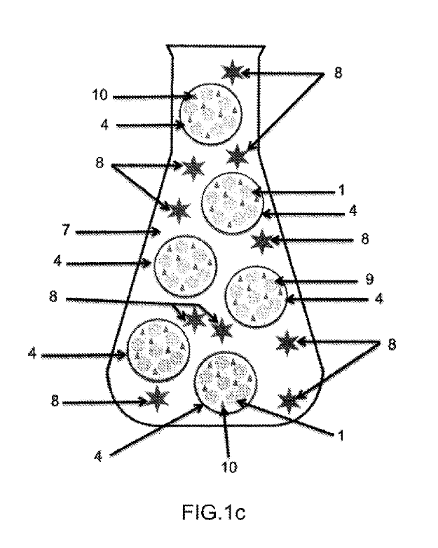

In FIG. 1a, there is schematically represented an inclusion complex 1 formed from a first active ingredient 2 as described above, and a cyclodextrin 3 as described above.

In FIG. 1b, there is schematically represented a portion of a microparticle 4 according to the invention which includes a matrix 5 of a biocompatible polymer as described above. The matrix 5 comprises pores 9 which contain inclusion complexes 1 such as the inclusion complex that is represented in FIG. 1a, as well as second hydrophilic active ingredients 6. Furthermore, second hydrophobic active ingredients 10 are solubilized in the matrix 5.

In FIG. 1c, there is schematically represented an aqueous suspension 7 according to the invention which contains microparticles 4 such as those that are represented in FIG. 1b, in addition to third active ingredients 8.

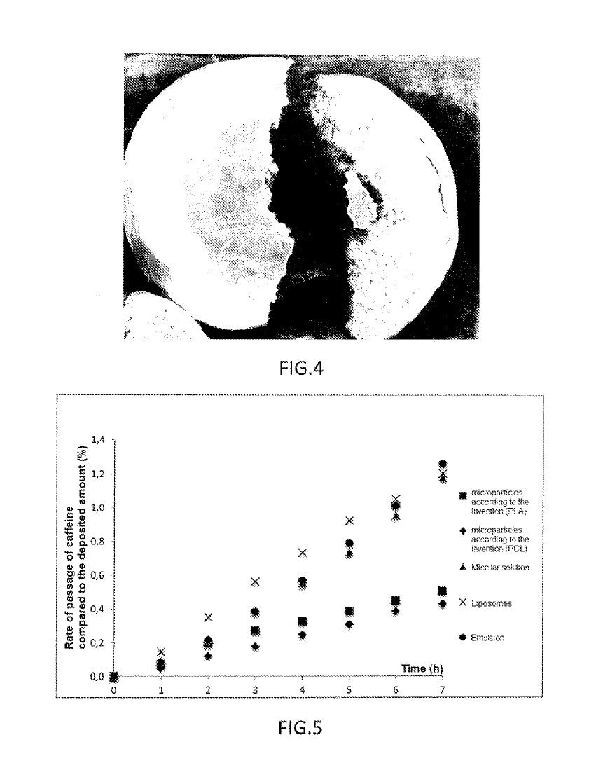

FIG. 4 is a photograph taken by scanning electron microscopy in which a microparticle according to the invention is visible, which microparticle has been cut into two halves. In this photograph, it is observed that the outer surface of the microparticle is smooth whereas the interior of the microparticle is porous. This photograph clearly shows that the microparticles according to the invention have a polymer matrix which includes closed pores. The apparatus that has been used for taking this photograph was a JEOL Neoscope JCM-5000 microscope.

1^{st} Experimental Part:

The diffusion of a first active ingredient, hesperidin, in the different layers of the skin has been assessed based on the manner by which it has been applied on the skin, namely:
1) in the case where the hesperidin has been encapsulated in microparticles according to the invention, and following Example 1 which has been detailed above,
2) in the case where the hesperidin has been encapsulated in microparticles of polymers,
3) in the case where the hesperidin has been provided in a free form,
4) in the case where the hesperidin has been provided in the form of an inclusion complex with a cyclodextrin.

More specifically, the hesperidin has been prepared in the following manner:
1) in the case where it has been encapsulated in microparticles according to the invention: This is the first example, the proportions of the constituents of which example at the different steps of the method according to the invention are detailed in Tables 1, 4 and 5 above;
2) in the case where it has been encapsulated in microparticles of polymers: the hesperidin has been encapsulated in microparticles of polymers which have been prepared in the same manner as the microparticles according to the invention, except that the 1^{st} aqueous solution did not contain cyclodextrins.
3) in the case where it has been provided in a free form: the hesperidin has been solubilized in water.
4) In the case where it has been provided in the form of an inclusion complex with a cyclodextrin: the hesperidin has been solubilized in water which has been saturated with β-cyclodextrins at a concentration of 18.5 g/L.

These 4 preparations have constituted 4 samples of cutaneous application of hesperidin. Hesperidin represented 0.5 weight % of the mass of each of these 4 samples.

Tests on these 4 samples have been carried out in accordance with the Directives: SCCS/1358/10 on date of Jun. 22, 2010 <<Basic criteria for the in vitro assessment of dermal absorption of cosmetic ingredient>>, OECD 28 of 2004. OECD being the abbreviation of <<Organization for Economic Co-operation and Development>> and SCCS being the abbreviation of <<Scientific Committee on Consumer Safety>>.

The material that has been used consisted of Franz cells. Skin explants, with an average surface area of 2.54 cm² and with a thickness comprised between 1.1 and 1.35 mm, have been used.

The experimental study has consisted in kinetics measurements over 24 and 48 hours, as well as a dismount of the cells after at the end of this time period, where all the layers of the skin have been separated through several biopsies in order to assess the distribution of hesperidin in these layers.

The hesperidin has been extracted until exhaustion from each cutaneous compartment.

The cumulated amounts (expressed in μg/cm²) in each layer of the skin have allowed assessment of the distribution of hesperidin, for each sample.

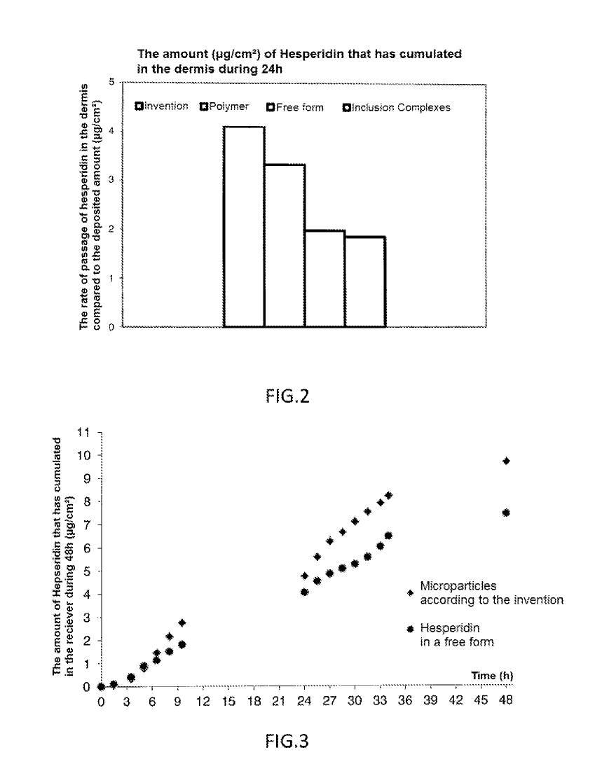

FIG. 2 is a diagram which details, for each sample, the amount of hesperidin that has cumulated in the dermis during 24 hours.

In the diagram of FIG. 2, there are represented, from the left to the right:
1) the results that have been obtained with the sample comprising microparticles according to the invention containing hesperidin;
2) the results that have been obtained with the sample comprising polymer microparticles containing hesperidin;
3) the results that have been obtained with the sample in the case where the hesperidin has been provided in a free form;
4) the results that have been obtained with the sample in the case where the hesperidin has been provided in the form of an inclusion complex with a cyclodextrin.

It is observed that the amounts of hesperidin that have cumulated in the dermis during 24 hours are as follows:
1) in the case where the hesperidin has been encapsulated in microparticles according to the invention: 4.11 μg/cm²,
2) in the case where the hesperidin has been encapsulated in microparticles of polymers: 3.32 μg/cm²,
3) in the case where the hesperidin has been provided in a free form: 1.98 μg/cm²,
4) in the case where the hesperidin has been provided in the form of an inclusion complex with a cyclodextrin: 1.85 μg/cm².

Thus, in light of the diagram of FIG. 2, it is observed that the encapsulation in microparticles according to the invention allows enhancing the diffusion of hesperidin in the dermis, in contrast to the other means that have been implemented and which consisted, respectively, of the encapsulation in microparticles of polymers, the free form and the inclusion complex with a cyclodextrin.

In particular, it is important to note that the microparticles according to the invention are more performant in provoking the accumulation of hesperidin in the dermis during 24 hours of exposure than microparticles of polymers which encapsulate the same tested active ingredient and which are known in the related art.

This diagram of FIG. 2 shows a synergistic effect between the hesperidin/cyclodextrin inclusion complex and its encapsulation in microparticles of polymers. This synergistic effect consists of an increase of the amount of hesperidin that cumulates in the dermis during 24 hours.

Thanks to the microparticles according to the invention, the bioavailability of the tested hesperidin in the dermis is the best. This is why it may be concluded that the action of the first active ingredients, which are intended to act in the dermis, will be reinforced when encapsulated in microparticles according to the invention.

Thus, this diagram of FIG. 2 clearly demonstrates two main benefits of the microparticles according to the invention on the skin:
the encapsulation at a first level, namely thanks to the formation of an inclusion complex in cyclodextrins which allows the first active ingredients to pass through the cutaneous barrier more easily, and
the encapsulation at a second level, namely in microparticles of polymers which allows accumulating the first active ingredients in the deep layers of the skin (namely the dermis).

The graph of FIG. 3 details the amount of hesperidin that has cumulated in the hesperidin-receiver liquid, as a function of time, from 0 to 48 hours:
in the case where the hesperidin has been provided in a free form;
in the case where the hesperidin has been encapsulated in microparticles according to the invention.

From the graph of FIG. 3, it is observed that:
As from the 6^{th} hour, and until the end of the experiment (namely, after 48 hours), the amount of hesperidin that has been retrieved in the receiver liquid is still much more significant in the sample where it has been contained in microparticles according to the invention than the sample where it has been provided in a free form.

As from the 6^{th} hour, the hesperidin diffuses much better in the different layers of the skin in the sample where it has been encapsulated in microparticles according to the invention than the sample where it has been provided in a free form.

In addition, it is observed, from the graph of FIG. 3, that the hesperidin penetration is not immediate in the case of the microparticles according to the invention. In fact, a latency time period is observed, that is to say a time period after which the hesperidin starts penetrating in the skin. This latency time period is about one hour and 30 minutes.

This delay of the penetration in the skin is explained by this dual level of encapsulation of the first active ingredient (namely, hesperidin in the context of the present experiment) which is contained in the microparticles according to the invention.

As a conclusion, this experimental part, which has been detailed above, demonstrates that the encapsulation of the cosmetic or dermatological active ingredient for topical use in microparticles according to the invention constitutes a quite performant means for vectorizing the penetration of active ingredients through the different layers of the skin, in particular until reaching the dermis, and this for a time period which may last for two days.

The dual-encapsulation of the microparticles according to the invention reveals all its interest:
a) the encapsulation at a first level by means of inclusion complexes formed from cyclodextrins which allows enhancing the accumulation of active ingredients in the different layers of the skin during the first hours after their application over the skin,
b) the encapsulation at a second level by means of the biocompatible polymer matrix which delays the penetration of the cosmetic or dermatological active ingredients for topical use.

2^{nd} Experimental Part:

Moreover, while using the same Franz cells and in accordance with the Directives that have been mentioned above in the 1^{st} experimental part, kinetics studies carried out over 7 hours and 24 hours have been conducted on other samples which are detailed below, in order to compare the rate of passage of three active ingredients (the tocopherol acetate, caffeine and the alpha-glucosyl-hesperidin) into the receiver liquid, depending on the galenic form in which they have been incorporated, said galenic forms were as follows:
microparticles according to a first embodiment of the invention (sample 1);
microparticles according to a second embodiment of the invention (sample 2)
a micellar solution (sample 3);
an emulsion (sample 4);
liposomes (sample 5).

The galenic forms of samples 3) to 5) correspond to galenic forms which are commonly used in the dermatological and dermatocosmetic field and which thereby constitute reference samples to be compared to samples 1) and 2) which consist of microparticles according to the present invention.

The preparation of the samples 1) to 5) of this 2^{nd} experimental part is described below:

Preparation of the Microparticles According to the Invention (Samples 1) and 2)):

The microparticles according to the first and second embodiments of the invention differed only but by the biocompatible polymer:
as regards the microparticles of sample 1), the biocompatible polymer was a PLA
as regards the microparticles of sample 2), the biocompatible polymer was a PCL.

The microparticles according to the invention have been obtained, for sample 1), in the following manner:

Steps a) to c) of the Method According to the Invention, Upon which Steps an Emulsion is Obtained:

| | | Weight |
|---|---|---|
| | | percentage |
| | | in the |
| PHASE | Ingredient | emulsion (%) |
| A | PLA | 33 |
| | dichloromethane | 50 |
| B | activated water: | 17 |
| | namely | |
| | alpha-glucosyl-hesperidin | 4 |
| | caffeine | 4 |
| | beta-cyclodextrins | 0.3 |
| | tocopherolacetate | 0.2 |
| | water | 8.5 |
| | Total: | 100.00 |

Table 9 detailing, for sample 1), the amounts of the constituents which are required for carrying out step a) of the method according to the invention.

Step d) of the Method According to the Invention, Upon which Step a Double Emulsion is Obtained:

| | | Percentage in the final double |
|---|---|---|
| PHASE | Ingredient | emulsion (%) |
| C | poly(vinyl alcohol) | 1 |
| | water | 88 |
| Emulsion | Emulsion | 11 |
| | namely | |
| | PLA | 3 |
| | dichloromethane | 6 |
| | activated water | 2 |
| | TOTAL | 100.00 |

Table 10 detailing the amounts of the constituents which are required for carrying out step d) of the method according to the invention.

Step e) of the Method According to the Invention (Namely the Extraction of Dichloromethane):

| | | Percentage in the final |
|---|---|---|
| PHASE | Ingredient | emulsion (%) |
| D | water | 75 |
| | isopropanol | 4 |
| Double | Double Emulsion | 21 |
| Emulsion | namely | |
| | poly(vinyl | 0.2 |
| | alcohol) | |
| | water | 18.6 |
| | PLA | 0.8 |
| | dichloromethane | 1.1 |
| | activated water | 0.4 |
| | TOTAL | 100.00 |

Table 11 detailing the amounts of the constituents which are required for carrying out step e) of the method according to the invention.

Step f) of the above-described method has been carried out and a sample 1) has been obtained, which sample was in the form of an aqueous suspension of microparticles according to a first embodiment of the invention.

The microparticles according to the invention have been obtained, for sample 2), in the following manner:

Steps a) to c) of the Method According to the Invention, Upon which Steps an Emulsion is Obtained:

| | | Weight |
|---|---|---|
| | | percentage in |
| | | theemulsion |
| PHASE | Ingredient | (%) |
| A | PCL | 41 |
| | dichloromethane | 41 |
| B | activated water: | 18 |
| | namely | |
| | alpha-glucosyl-hesperidin | 4 |
| | caffeine | 4 |
| | beta-Cyclodextrins | 0.3 |
| | tocopherolacetate | 0.2 |
| | water | 9.5 |
| | Total: | 100.00 |

Table 12 detailing, for sample 2), the amounts of the constituents which are required for carrying out step a) of the method according to the invention.

Step d) of the Method According to the Invention, Upon which Step a Double Emulsion is Obtained:

| | | Percentage in the final double |
|---|---|---|
| PHASE | Ingredient | emulsion (%) |
| C | poly(vinyl alcohol) | 1 |
| | water | 88 |
| Emulsion | Emulsion | 11 |
| | namely | |
| | PCL | 4.5 |
| | dichloromethane | 4.5 |
| | activated water | 2 |
| | TOTAL | 100.00 |

Table 13 detailing the amounts of the constituents which are required for carrying out step d) of the method according to the invention.

Step e) of the Method According to the Invention (Namely the Extraction of Dichloromethane):

| | | Percentage in the final |
|---|---|---|
| PHASE | Ingredient | emulsion (%) |
| D | water | 75 |
| | isopropanol | 4 |
| Double | Double Emulsion | 21 |
| Emulsion | namely | |
| | poly(vinyl | 0.2 |
| | alcohol) | |
| | water | 18.6 |
| | PCL | 0.95 |
| | dichloromethane | 0.95 |
| | activated water | 0.4 |
| | TOTAL | 100.00 |

Table 14 detailing the amounts of the constituents which are required for carrying out step e) of the method according to the invention.

Step f) of the above-described method has been carried out and a sample 2) has been obtained, which sample was in the form of an aqueous suspension of microparticles according to a first embodiment of the invention.

Thus, the microparticles of samples 1) and 2) comprise:
the alpha-glucosyl-hesperidin, which is a first active ingredient of said microparticles, as it forms inclusion complexes with the cyclodextrins. In fact, as has been explained above, the alpha-glucosyl-hesperidin is a hydrophilic active ingredient which however includes a hydrophobic portion which allows the formation of an inclusion complex with a cyclodextrin.
caffeine, which is a second ingredient of said microparticles. Caffeine does not form inclusion complexes with the cyclodextrins as it does not contain, in its chemical structure, a hydrophobic portion which would be appropriate for forming an inclusion complex with a cyclodextrin. In fact, the hydrophobic portion of its chemical structure is too small to form interactions with the cyclodextrins that are strong enough. Therefore, there is no formation of inclusion complexes between a cyclodextrin and caffeine. This second active ingredient is also hydrophilic and it is present in the pores of said microparticles.
tocopherol acetate, which is a second active ingredient of said microparticles in the sense of the present invention. This second ingredient is hydrophobic and it is present in the biocompatible polymer matrix of said microparticles (more specifically, PLA for the microparticles of sample 1 and PCL for the microparticles of sample 2).

Preparation of the Micellar Solution (Sample 3):

The micellar solution was a mixture comprising a surfactant, which is designated according to INCI as <<Oleth-20>> (also known under the designation of Polyoxyethylene (20) Oleyl Ether), in water at a concentration of 4 weight %.

INCI is the abbreviation of <<International Nomenclature of Cosmetic Ingredients>>.

Preparation of the Emulsion (Sample 4):

The emulsion was a mixture comprising:
20 weight % of isononylisononanoate;
6 weight % of the <<Oleth-20>> surfactant;
0.5 weight % of a Xanthan gum gallant;
qs water.

Preparation of the Liposomes (Sample 5):

The liposomes have been constituted from a mixture comprising:
35 weight % of Natipide® II (a product that is commercialized by the company Lipoid and which comprises water, lecithin and ethanol);
65 weight % of water

In order to make it possible to compare the release of these three active ingredients, which ingredients have been detailed above, based on the chosen galenic form (namely, the microparticles according to the invention, the micellar solution, the emulsion, the liposomes), the mass proportions of these active ingredients have been properly adjusted so that in all samples 1) to 5), they were as follows:
tocopherol acetate (hydrophobic active ingredient): 0.2 weight % in the organic phase of the considered sample;
caffeine and alpha-glucosyl-hesperidin (hydrophilic active ingredients): respectively, 0.5 weight % in the aqueous phase of the considered sample.

Only the microparticles according to the invention contained inclusion complexes. In fact, the alpha-glucosyl-hesperidin has been complexed with the cyclodextrins. In all the other galenic forms, there were no cyclodextrins, and therefore, there were no inclusion complexes.

The conclusions of the studies that have been conducted on samples 1) to 5) are exposed below.

Conclusions of the Study Regarding the Release of the Combination of Active Ingredients Over 7 Hours:

FIG. 5 shows, for samples 1) to 5), the kinetics of the caffeine penetration over 7 hours.

In fact, FIG. 5 is a graph of the measurements of the rate of passage of the active ingredient (in this instance, caffeine) into the receiver liquid, that is to say the ratio of the cumulated amount of permeated caffeine to the deposited amount (expressed as a percentage) as a function of time, and this for samples 1) to 5).

In light of FIG. 5, it is observed that the release of caffeine (namely, a hydrophilic active ingredient which is present in the pores of the microparticles according to the invention and which forms no inclusion complexes with the cyclodextrins) is delayed thanks to the microparticles according to the invention, in contrast to the other galenic forms (samples 3) to 5)).

In fact, in FIG. 5, it is observed that after 7 hours, the rate of passage of caffeine from the microparticles according to the invention into the receiver liquid is lower than the rates of passage of the caffeine of the emulsion, the liposomes and the micellar solution which, in this regards, are fairly close to each other.

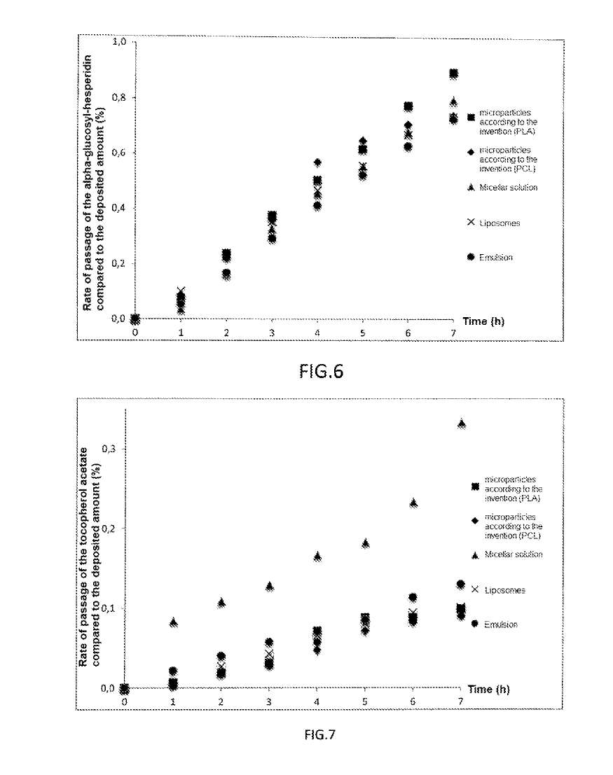

FIG. 6 shows, for samples 1) to 5), the kinetics of the penetration of the alpha-glucosyl-hesperidin, tested under the different galenic forms, over a time period of 7 hours. In fact, FIG. 6 is a graph of the measured rate of passage of this other active ingredient, which is the alpha-glucosyl-hesperidin, into the receiver liquid, that is to say the ratio of the cumulated amount of permeated alpha-glucosyl-hesperidin to the deposited amount (expressed as a percentage), as a function of time.

In light of FIG. 6, the release of the alpha-glucosyl-hesperidin (namely, a hydrophilic active ingredient which has formed inclusion complexes with the cyclodextrins in the microparticles according to the invention) is accelerated thanks to the polymer microparticles according to the invention, in contrast to the other galenic forms (samples 3) to 5)).

In fact, in FIG. 6, it is observed that after 7 hours, the rate of passage of the alpha-glucosyl-hesperidin from the microparticles of sample 1) according to the invention, the biocompatible polymer of which microparticles is PLA, into the receiver liquid is almost equal to, or still slightly higher than, the rates of passage of the alpha-glucosyl-hesperidin from the emulsion, the liposomes and the micellar solution (namely, the comparative samples 3) to 5)).

The results that are expressed in FIGS. 5 and 6 clearly highlight the difference in the release of the two hydrophilic active ingredients which are comprised within the microparticles according to the invention.

Note that, depending on the desired application to which these microparticles according to the invention are intended, it is possible to choose the rate of release of the active ingredients, that is to say, whether this release takes place in a delayed or in an accelerated manner.

Thus, in the case where it is desired to promote the penetration of a given active ingredient, suitable physicochemical conditions would be implemented in order that this active ingredient forms complexes with the cyclodextrins in the microparticles according to the invention.

FIG. 7 shows, for the different galenic forms that have been tested, the kinetics of the penetration of the tocopherol acetate, over a time period of 7 hours. In fact, FIG. 7 details, for samples 1) to 5), the rate of passage of this other active ingredient, which is the tocopherol acetate, into the receiver liquid, that is to say the ratio of the cumulated amount of permeated tocopherol acetate to the deposited amount (expressed as a percentage), as a function of time.

In light of FIG. 7, the release of the tocopherol acetate (namely, a hydrophobic active ingredient) from the microparticles according to the invention is maintained at the same level as the conventional galenic formulations which are the liposomes and the emulsion.

In fact, in FIG. 7, it is observed that after 7 hours, the rate of passage of the tocopherol acetate from the microparticles according to the invention into the receiver liquid is almost equal to the rate of passage of the tocopherol acetate of the emulsion and of the liposomes (namely, the comparative samples 4) and 5)).

Furthermore, because of its hydrophobic nature and the physicochemical composition of the cutaneous barrier, the release of the tocopherol acetate is lower than the release of the hydrophilic active ingredients which are caffeine and the alpha-glucosyl-hesperidin.

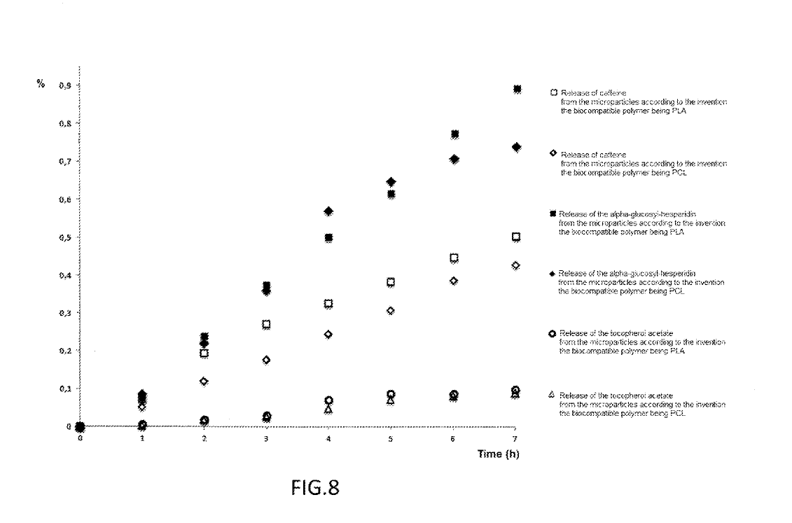

FIG. 8 is a graph detailing, for samples 1) and 2) (namely, the samples comprising microparticles according to the invention), the rates of passage of the three active ingredients that have been tested and which are caffeine, the alpha-glucosyl-hesperidin and the tocopherol acetate compared to their respective deposited amounts (expressed in %) as a function of time.

In FIG. 8, it is observed that after 7 hours, with the microparticles according to the invention, the rate of passage of the tocopherol acetate (a hydrophobic active ingredient) into the receiver liquid is lower than the rate of passage of caffeine (a hydrophilic active ingredient which is present in the pores of said microparticles and which does not form inclusion complexes with the cyclodextrins) which is, in turn, lower than the rate of passage of the alpha-glucosyl-hesperidin (a hydrophilic active ingredient which does form inclusion complexes with the cyclodextrins of said microparticles).

These differences in the rate of passage into the receiver liquid demonstrate that the different active ingredients that are comprised within the microparticles according to the invention are not released in the skin at the same speed.

In light of the results that are detailed in these FIGS. 5 to 8, it is observed that the mircoparticles according to the invention present release profiles in the skin which are original and which make the present invention modulable, to the extent that the effects of the cutaneous penetration of the active ingredients, when encapsulated in said microparticles, can be modulated as desired.

In addition, for the microparticles according to the invention, whether said microparticles comprised a PLA or a PCL biocompatible polymer, no significant difference has been observed as regards the release, in the skin, of the three active ingredients that have been tested and which were caffeine, the alpha-glucosyl-hesperidin and the tocopherol acetate.

Conclusions of the Comparison of the Cumulated Amounts of the Active Ingredients that have Cumulated in the Receiver Liquid after 24 Hours:

A last assay of the released amounts of the tested active ingredients has been performed after 24 hours, for samples 1) to 5).

The amount that has cumulated over the duration of this 2^{nd} experimental part, that is to say over 24 hours, has been calculated and related to the initially deposited amount of active ingredient. Thus, is obtained the rate of passage, in the skin, of the active ingredients compared to the deposited amounts, or in other words, the ratio of the cumulated amount of said permeated active ingredients to their deposited amounts (expressed as a percentage).

In order to compare the obtained percentages, it has been convenient to relate the rate of passage of caffeine (namely, the hydrophilic active ingredient which has not been complexed with the cyclodextrins in the microparticles according to the invention) and the rate of passage of the alpha-glucosyl-hesperidin (namely, the hydrophilic active ingredient which has been complexed with the cyclodextrins in the microparticles according to the invention), to the rate of passage of the tocopherol acetate (namely, the hydrophobic active ingredient).

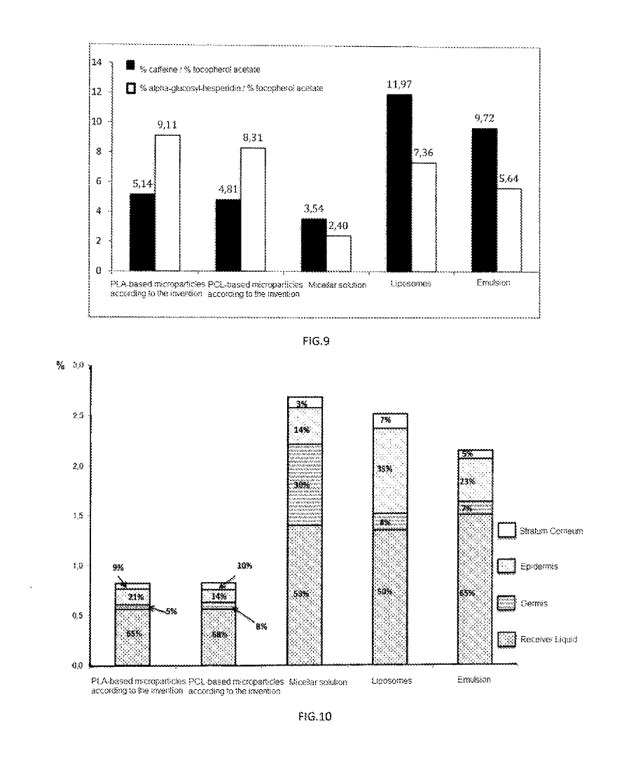

These coefficients are referred to as <<relative permeation rates>>. They are represented in FIG. 9.

In light of the diagrams of FIG. 9, the effect of the cyclodextrins in the microparticles according to the invention is clearly highlighted.

More specifically, in the two diagrams regarding the microparticles according to the invention, the relative permeation rate of the hydrophilic active ingredient that has been complexed with the cyclodextrins (namely, the alpha-glucosyl-hesperidin) is almost twice high as the relative permeation rate of the active ingredient that has not been complexed with the cyclodextrins (namely, caffeine).

In addition, in comparison with the other samples 3) to 5) (that is to say the other galenic forms of these tested active ingredients which are the micellar solution, the emulsion and the liposomes), there is observed, for the microparticles according to the invention, a trend reversal between each of the two ratios.

Thus, the cyclodextrins that are contained in the microparticles according to the invention allow overcoming the slow-down of the permeation of a hydrophilic active ingredient (such as for example, caffeine), which slow-down is specific to the encapsulation as has been mentioned in the kinetics that have been represented in FIGS. 5 to 8. Hence, the benefit of the presence of cyclodextrins within the microparticles according to the invention is to allow for an enhancement of the permeation of a hydrophilic active ingredient. Its permeation is almost at the same magnitude as the permeation of the other galenic forms of samples 3 to 5, in which, recall, the hydrophilic active ingredient has not been encapsulated nor has it been complexed, and therefore, it has no macromolecular barrier that would have delayed its permeation.

This demonstrates the specific nature of the release of the microparticles according to the invention in comparison with the galenic forms that are conventionally used in the dermatological and dermatocosmetic fields.

In addition, the results of this 2^{nd} experimental part demonstrate that the cyclodextrins that are present in the microparticles according to the invention provide a real benefit when it comes to the penetration of a hydrophilic active ingredient.

Thus, for the hydrophilic active ingredients, the release profile of the microparticles according to the invention stands out from the other galenic forms of samples 3) to 5).

Conclusions of the Study Regarding the Penetration of the Active Ingredients in the Different Layers of the Skin after 24 Hours:

Afterwards, at the end of 24 hours of experimentation, the Franz cells have been dismounted and biopsies have been carried out in order to assay the active ingredients that were present in each of the layers of the skin.

Thus, it has been possible to directly compare the galenic forms of samples 1) to 5) to each other, in order to set out the specificities of the microparticles according to the invention in comparison with the other so-called comparative galenic forms, but also the specificities of the release of the active ingredients when compared to each other.

FIG. 10 represents, for samples 1) to 5), the diagrams of the rates of passage (expressed as percentages) compared to the applied dose of caffeine (namely, the hydrophilic active ingredient which has not been complexed with the cyclodextrins in the microparticles according to the invention) at the end of a time period of 24 hours, as well as the distribution (expresses as percentages) of this active ingredient in the different layers of the skin and in the receiver liquid.

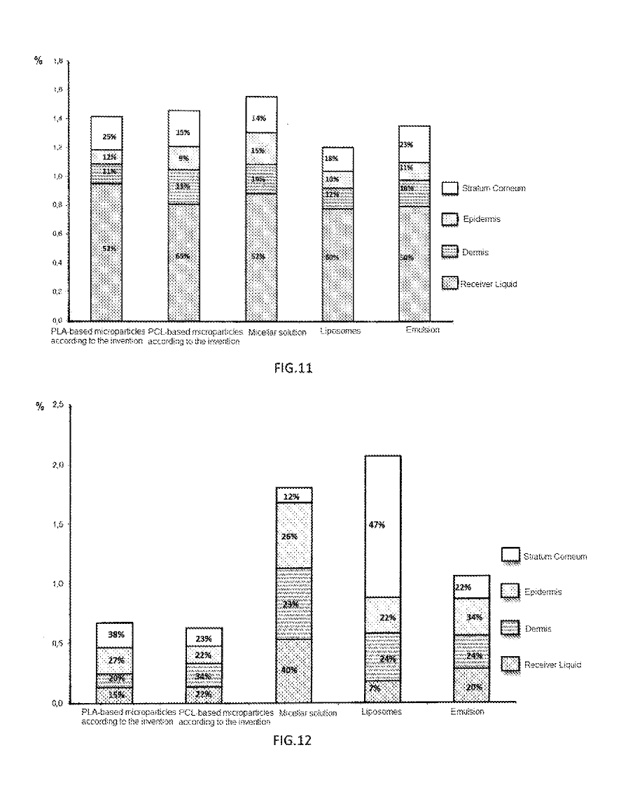

FIG. 11 represents, for samples 1) to 5), diagrams of the rates of passage (expressed as percentages) compared to the applied dose of the alpha-glucosyl-hesperidin (namely, the hydrophilic active ingredient which has been complexed with the cyclodextrins in the microparticles according to the invention) at the end of a time period of 24 hours, as well as the distribution (expresses as percentages) of this active ingredient in the different layers of the skin and in the receiver liquid.

FIG. 12 represents, for samples 1) to 5), diagrams of the rates of passage (expressed as percentages) compared to the applied dose of the tocopherol acetate (namely, the hydrophobic active ingredient) at the end of a time period of 24 hours, as well as the distribution (expresses as percentages) of this active ingredient in the different layers of the skin and in the receiver liquid.

In light of the results that are represented in FIG. 10, it is observed that caffeine (the hydrophilic active ingredient which has not been complexed with the cyclodextrins) of the microparticles according to the invention does not penetrate much in the layers of the skin. In fact, according to FIG. 10, the rates of passage of this active ingredient from the microparticles according to the invention toward the layers of the skin are lower than the rates of passage of this active ingredient from the other so-called comparative galenic forms which are the emulsion, the liposomes and the micellar solution.

In addition, in light of FIG. 11, the alpha-glucosyl-hesperidin (namely, the hydrophilic active ingredient which has been complexed with the cyclodextrins in the microparticles according to the invention) of the microparticles according to the invention presents a rate of passage which is equivalent to those of the alpha-glucosyl-hesperidin of the comparative galenic formulations of samples 3) to 5).

Thus, in the microparticles according to the invention, the cyclodextrin allows conveying considerable amounts of active ingredients, in particular hydrophilic active ingredients, in the dermis and in the epidermis.

In light of the results that are represented in FIGS. 10 and 11, and in comparison with the comparative galenic formulations of samples 3) to 5, it is observed that in the microparticles according to the invention, the penetration of the hydrophilic active ingredient that has not been complexed with the cyclodextrins is considerably slowed because of the encapsulation in the polymer matrix. This slow-down of the penetration is not observed with the active ingredient that has been complexed with the cyclodextrins.

Thus, in the microparticles according to the invention, the encapsulation in the polymer matrix slows the diffusion of a hydrophilic active ingredient that has not been complexed with the cyclodextrins.

FIGS. 10 and 11 demonstrate that the two hydrophilic active ingredients that have been tested, and which were comprised within the microparticles according to the invention, have quite different behaviors when compared to each other but also when compared to the comparative galenic formulations of samples 3) to 5).

In addition, in light of the results of FIG. 12, which results have been obtained with the microparticles according to the invention, it is observed that the tocopherol acetate (namely, the hydrophobic active ingredient) does not penetrate much. In fact, the rates of passage of this active ingredient from the microparticles according to the invention are lower than the rates of passage of this active ingredient from the other gale nic forms that have been tested, since the release of this active ingredient, which has been encapsulated in the microparticles according to the invention, is controlled.

Furthermore, in contrast to the liposomes, the accumulation of this hydrophobic active ingredient in the stratum corneum is avoided. Besides, in contrast to the micellar solution, the penetration of this hydrophobic active ingredient into the receiver liquid is also avoided.

The encapsulation of this hydrophobic active ingredient in the microparticles according to the invention allows targeting the dermis and the epidermis better than the other galenic forms which have been tested with samples 3) to 5).

Thus, the technical benefit provided by the microparticles according to the invention and which has already been mentioned above is again observed, namely the possibility of modulating the passage of the hydrophilic active ingredient depending on whether or not it forms inclusion complexes with the cyclodextrins. The interest of the cyclodextrins as a promoter of the penetration of a hydrophilic active ingredient is again demonstrated with these FIGS. 10 to 12.

Finally, the interest of the microparticles according to the invention lies in the stabilization of the active ingredients in the cosmetic product.

Thus, the microparticles according to the invention offer the following technical advantages:
the possibility of encapsulating several active ingredients of different natures (hydrophilic and hydrophobic);
these active ingredients are stabilized by the protection of the biocompatible polymer matrix comprised within the microparticles according to invention. This protection of the biocompatible polymer matrix slows the diffusion of these active ingredients in the skin.

Furthermore, the results of this 2^{nd} experimental part demonstrate that it is possible to modulate the diffusion of the hydrophilic active ingredients thanks to the cyclodextrins that are contained in the microparticles according to the invention. In fact, it is possible to choose whether it is desirable or not to promote the penetration of these active ingredients.

The cyclodextrins allow the active ingredient, which forms inclusion complexes with these cyclodextrins, to release at the same depth as the comparative galenic forms of samples 3) to 5).

The penetration and the distribution of the active ingredient, which has been complexed with the cyclodextrins, in the layers of the skin are also similar to the comparative galenic forms when considering the ratio of the total amount of the active ingredient that has penetrated to the applied dose.

The penetration of a hydrophobic active ingredient is modulated thanks to the microparticles according to the invention which ensure a slow diffusion and a balanced distribution between the layers of the skin.

1. A microparticle which includes a solid and porous matrix, said matrix comprising at least one biocompatible polymer and including, within its pores, water and at least one inclusion complex formed between a cyclodextrin and at least one first active ingredient, wherein the first active ingredient is selected in the group constituted by cosmetic active ingredients and dermatological active ingredients for topical use and said microparticle further comprises at least one second cosmetic or dermatological active ingredient for topical use which is not in the form of an inclusion complex with the cyclodextrin.

2. The microparticle according to claim 1, wherein the pores that the matrix includes are closed.

3. The microparticle according to claim 1, wherein said second ingredient is a hydrophilic active ingredient which is contained in the pores of the and/or a second hydrophobic active which is matrix and/or a second hydrophobic active ingredient which is solubilized in the matrix.

4. The microparticle according to claim 1, wherein it comprises a plurality of inclusion complexes formed from a plurality of cyclodextrins which are identical or different from each other and a plurality of first active ingredients which are identical or different from each other.

5. The microparticle according to claim 1, wherein the first active ingredient and the second active ingredient are cosmetic ingredients which are selected in the group constituted by the active ingredients having an anti-ageing effect, an anti-wrinkle effect, an anti-redness effect, a moisturizing effect, a soothing effect, a brightening effect, a plumping effect or still a purifying effect.

6. The microparticle according to claim 1, wherein the first active ingredient is selected in the group constituted by hesperidin, the hesperidin derivatives, the lipoic acid and the lipoic acid derivatives.

7. The microparticle according to claim 1, wherein the second active ingredient is selected in the group constituted by tocopherol acetate, the pyrrolidone carboxylic acid, caffeine, amino acids, peptides, oligosaccharides, polysaccharides, menthol, methyl nicotinate, unsaturated fatty acids, retinol, tocopherol and their derivatives.

8. An aqueous suspension, wherein it comprises microparticle according to claim 1.

9. The aqueous suspension according to claim 8, wherein it further comprises at least one third active ingredient selected in the group constituted by cosmetic active ingredients and dermatological active ingredients for topical use.

10. A cosmetic or dermatological composition for topical use, wherein it comprises at least microparticles according to claim 1.

11. A cosmetic or dermatological composition for topical use, wherein it comprises at least one aqueous suspension according to claim 8.

12. A method for producing microparticle according to claim 3, wherein it comprises the following steps consisting in:
a) preparing a first aqueous solution which comprises at least one first active ingredient selected in the group constituted by the cosmetic active ingredients and the dermatological active ingredients for topical use, and at least one cyclodextrin, the amounts of the first active ingredient and the cyclodextrin being determined so that the first active ingredient and the cyclodextrin form an inclusion complex, said first aqueous solution further comprises at least one second active ingredient, in the case where the second ingredient is a hydrophilic ingredient;
b) preparing a second organic solution which comprises at least one organic solvent and at least one biocompatible polymer, the organic solvent solubilizing the biocompatible polymer, said second organic solution further comprises at least one second active ingredient, in the case where the second active ingredient is a hydrophobic ingredient;
c) introducing the first aqueous solution in the second organic solution and stirring this set, which set results from the combination of these two solutions, so as to obtain an aqueous-in-organic type emulsion;
d) introducing this emulsion which has been obtained upon completion of step c) in a third aqueous solution and stirring this set, which set results from the combination of the emulsion and the third solution, so as to obtain an aqueous-in-organic-in-aqueous type double emulsion;
e) extracting the organic solvent by means of a water-soluble organic co-solvent;
f) evaporating the organic solvents and co-solvents, so as to obtain an aqueous suspension of the microparticles;
g) optionally, recovering the microparticles.
